(19) United States
(12) Patent Application Publication
(10) Pub. No.: US 2020/0030253 A1
(43) Pub. Date: Jan. 30, 2020

(54) Methods and compositions for treatment of body conditions

(71) Applicant: Mohammad Ali Kharazmi, Corona Del Mar, CA (US)

(72) Inventor: Mohammad Ali Kharazmi, Corona Del Mar, CA (US)

(21) Appl. No.: 16/523,967

(22) Filed: Jul. 26, 2019

Related U.S. Application Data
(60) Provisional application No. 62/703,813, filed on Jul. 26, 2018.

(51) Int. Cl. A61K 31/047; A61K 9/127; A61K 47/42; A61K 47/24; A61K 9/00; A61K 35/28; A61K 35/50

(52) U.S. Cl. CPC A61K 31/047; A61K 9/127; A61K 47/42; A61K 35/50; A61K 9/0014; A61K 9/0019; A61K 35/28; A61K 47/24

(57) ABSTRACT

In embodiments, the invention includes treatment compositions for treatment of wounds and body dysfunction due to injury, aging, degeneration, obesity, or disease. It includes compositions and methods of administering the compositions. The compositions include cannabidiol and either human stem cells or stem cell products.

### RELATED PATENT APPLICATION

This application claims the benefit of U.S. provisional patent application No. 62/703,813 entitled Methods and compositions for treatment of body conditions, filed Jul. 26, 2018 by Mohammad Ali Kharazmi. The entire content of this provisional patent application is incorporated by reference herein for all purposes.

### BACKGROUND

### Field

This invention is in the field of treatment of wounds and body dysfunction due to injury, aging, degeneration, obesity, or disease. It includes compositions and methods of administering the compositions. The compositions include cannabidiol and either human stem cells or stem cell products. The compositions improve the condition of damaged tissues. Damage includes cuts, abrasions, wounds, burns, scars, broken or fractured bones, blemished skin, or other conditions.

Tissue may be damaged by trauma, wound, burn, surgery, disease, compromised circulation, infarct, degeneration, aging, and other events or conditions. Tissues may also suffer environmental or disease-induced damage such as peripheral neuropathy and retinal overgrowth. Damaged tissue may heal naturally, but this process may be slow or incomplete. In some cases, the natural healing process produces a functional deficit such as noncontractile segment of muscle or a cosmetic defect such as a heavy scar.

Healing of damaged or dysfunctional tissue may be impaired by a variety of insults including aging, injury, circulatory impairment, and nerve damage. As tissue ages, hormone levels change and the structure of tissue often thins and weakens. Tissue fibroblasts may become less active and tissues becomes thinner and lose elasticity; as tissue thins, circulation can lessen and innervation may become sparser or less responsive. Tissue can show a decrease in volume and elasticity. There is a need to prevent and reverse these conditions to maintain and recover healthy function.

The body's ability to repair such damage varies by the tissue involved. Repair is generally imperfect and declines with age. For example, an infant can regrow a severed fingertip, but, absent special treatment, an adult cannot.

Natural healing may be promoted by a variety of treatments, which range from simple absorbent and antimicrobial application to surgical grafts, placement of resorbable scaffolds, and implant of exogenous stem cells.

The normal process of healing goes through a series a stages, illustrated here by that for wounded skin. In the wound healing process, injured tissue is repaired, specialized tissue is regenerated, and new tissue is reorganized. Three major phases are an inflammation stage of zero to three days, a proliferation stage of three to twelve days, and a remodeling phase of a few days to six months or more. In the inflammation phase, platelet aggregation and clotting form a matrix which traps plasma proteins and blood cells and induces the in-migration of various cells from surrounding tissues. In the cellular proliferation phase, new connective or granulation tissue and blood vessels form. In the remodeling phase, granulation tissue is replaced by a network of collagen and elastin fibers producing scar tissue. Each of these phases is at least partly dependent on signaling by growth factors produced by healing tissue and by adjacent tissue.

Tissue includes both cellular and extracellular components (including a structured extracellular matrix), so tissue repair requires at least some replication-competent cells to repopulate the damaged area. Many of these cells may be fibroblasts, which also secrete much of the extracellular matrix. Fibroblasts alone are insufficient to regenerate damaged skin or other tissue at least in part because they are end-stage cells with capabilities defined by development lineage. They are incapable of generating many other tissue structures, such as follicles and sebaceous glands in skin, cartilage, dentin, enamel, or bone in the skeleton, or most of the wide variety of tissue types of the body. However, other reservoirs of cells capable of regenerating at least some of these structures exist in tissue. For example, skin contains reservoirs of dermal stem cells in areas surrounding a wound; these are capable of regenerating the more complex structural elements. Snippert et al. reported in Science 327 p 1385 2010 that mammalian epidermis includes three self-renewing compartments: the hair follicle, the interfollicular epidermis, and the sebaceous gland. Adult dermal stem cells repaired wound damage, including the formation of new hair follicles. Dermal stem cells that are positive for a leucine-rich repeat containing G protein coupled receptor 6 (Lgr6+) produced interfollicular epidermis and sebaceous glands. Cells bearing Lgr5, a related G protein coupled receptor, regenerated follicular cells.

One approach to improve natural healing is to treat the damaged tissue with one or more cellular growth factors. Many tissues respond to mixtures of growth factors to encourage regeneration. In some cases, the growth factors may be produced by a cell culture as a conditioned medium. For example, US 2012/0065129 A1 to Park et al. asserts that a culture medium of adipose-derived stem cells and growth factors isolated from the culture medium can be advantageously applied for wound healing. The applied growth factors are intended to recruit and support replication-competent cells to repair the damage. A difficulty with this approach is that stem cells may produce a mix of products that are not appropriate to a particular wound environment. In some cases stem cell products may include materials (such as pro-inflammatory cytokines during the proliferation stage or the remodeling phase) that may interfere with further healing. An appropriate mix of growth factors should recruit, support, and activate a mix of cell types that produces improved healing outcomes. There is thus a need for a growth factor composition that provides such a mix of cell products and supports improved healing outcomes.

Further, exogenous stem cells may form a useful part of treatment composition to take part in improved healing outcomes when endogenous stem cells are insufficient. Such exogenous stem cells may produce a mix of cell products that may not comport with optimal healing. There is thus a need for a stem cell containing treatment composition that regulates the production of cell products from the stem cells to supports improved healing outcomes.

Injury to and healing of tissue damage can be painful and the surrounding tissue may be damaged by the inflammation of treatment regime itself. There is thus a need for a treatment composition that relieves such pain.

### SUMMARY

In embodiments, the invention includes treatment compositions and method of treatment using the compositions.

In one embodiment, the treatment composition includes a human stem cell, a first amount of cannabidiol, and an aqueous carrier. The human stem cell may include one or more of a hematopoietic stem cell, an endothelium-derived stem cell, adipose-derived stem cell, a tooth mesenchyme-derived stem cell, or a placenta-derived stem cell. The cannabidiol may be associated with a co-solubilizing agent, which may be one or more of a lipophilic surfactant or a protein.

In an embodiment, co-solubilizing agent may include one or more of dipalmitoylphosphatidylcholine or phosphatidylinositol, and the cannabidiol may be packaged in a liposome.

In other embodiments, the invention includes a treatment composition including a conditioned medium from a human stem cell culture and a first amount of cannabidiol. The conditioned medium may be derived from a culture admixed with a second amount of cannabidiol. The second amount of cannabidiol may be associated with a co-solubilizing agent. The human stem cell culture may include one or more of a hematopoietic stem cell, an endothelium-derived stem cell, adipose-derived stem cell, a tooth mesenchyme-derived stem cell, or a placenta-derived stem cell.

In some embodiments, the first amount of cannabidiol may be associated with a co-solubilizing agent. The co-solubilizing agent may include one or more of a lipophilic surfactant or a protein.

The co-solubilizing agent may include one or more of dipalmitoylphosphatidylcholine or phosphatidylinositol, and the cannabidiol may be packaged in a liposome.

In other embodiments, the invention includes a method of treating a body dysfunction that includes administering any of the above described compositions to an affected area of a human.

The step of administering may include a topical application of the composition. In some embodiments, the composition further includes a thickening agent.

In other embodiments, the step of administering includes an injection of the composition to an affected area of a human.

### DETAILED DESCRIPTION

The invention includes methods of treating injuries or dysfunction and compositions useful in such methods. In embodiments, the compositions include cannabidiol and either living stem cells or conditioned stem cell culture medium. In other embodiments, the invention includes methods treating an affected area of a human with one or more of the compositions.

### Cannabidiol

Cannabidiol is the major nonpsychoactive ingredient in cannabis. It is reported to possess neuroprotective and anti-inflammatory effects. The major psychotropic component of cannabis, Δ⁹-THC, activates the endocannabinoid system, which consists of receptors, synthetic and degradative enzymes, and transporters. Δ⁹-THC binds to two G-protein-coupled cell membrane receptors, consequently named the G-protein-coupled cannabinoid (CB) cannabinoid type 1 (CB₁) and type 2 (CB₂) receptors, to exert its effects. Endogenous lipophilic ligands (endocannabinoids) including anandamide and 2-arachidonoylglycerol also bind CB₁ and CB₂. CB₁ receptors are found primarily in the brain but also in several peripheral tissues. CB₂ receptors are mainly found in immune and hematopoietic cells, but can become upregulated in other tissues.

Cannabidiol has low affinity for CB₁ and CB₂ receptors, but appears to act as both an agonist and antagonist of CB₂ receptors depending on concentration. This paradoxical action is likely due to indirect action on other receptors or enzymes that are functionally linked to the CB₂ receptor. Cannabidiol interacts with many other, non-endocannabinoid signaling systems. At low micromolar to sub-micromolar concentrations, cannabidiol blocks equilibrative nucleoside transporter (ENT), the orphan G-protein-coupled receptor GPR55, and the transient receptor potential of melastatin type 8 (TRPM8) channel. Conversely, cannabidiol enhances the activity of the 5-HT₁ₐ receptor, the α3 and α1 glycine receptors, the transient receptor potential of ankyrin type 1 (TRPA1) channel, and has a bidirectional effect on intracellular calcium. At higher micromolar concentrations, cannabidiol activates the nuclear peroxisome proliferator-activated receptor-γ and the transient receptor potential of vanilloid type 1 (TRPV1) and 2 (TRPV2) channels while also inhibiting cellular uptake and fatty acid amide hydrolase-catalyzed degradation of anandamide. Cannabidiol is a potent antioxidant because of its multiple phenolic structures.

Cannabidiol has high lipophilicity (K_{octanol-water} ˜6-7) and consequently very low water solubility. This limits its availability in many formulations. In embodiments, the invention increases the effective solubility of cannabidiol by associating the cannabidiol with other agents. While these other agents may be simple hydrophobic solvents such as octanol, such solvents are also sparingly soluble in water unless emulsified. In embodiments, cannabidiol is co-solubilized by mixing into liposomes containing one or more of lipophilic surfactants such as dipalmitoylphosphatidylcholine or phosphatidylinositol. Cholesterol may be added as a further liposome component to improve stability. Dipalmitoylphosphatidylcholine is a phospholipid consisting of two palmitic acids attached of a phosphatidylcholine head-group. Phosphatidylinositol is a phosphatidylglyceride including an inositol group. These materials are merely illustrative of a class of lipophilic surfactants such as occur in cell membranes. These materials have been reported to be useful to prepare liposomes containing cannabidiol (see, for example, Hung, et al. PCT publication WO 01/03668, incorporated by reference for its teaching of liposome encapsulation of cannabinoids). One or more of these lipophilic surfactant material (or a mixture of the materials with cholesterol) may be mixed with cannabidiol in organic solvent with cannabidiol forming between 0.5 and 10% of the weight of the mixture. After drying the solvent the residue may be mixed with phosphate buffered saline (120 mM, pH 7) and extruded through 400 nm pore sized polycarbonate filters to form liposomes.

In some embodiments, a lower concentration of dipalmitoylphosphatidylcholine or phosphatidylinositol may be used so that each cannabidiol molecule is paired with one to five lipophilic surfactant molecules below the critical micellar concentration and without extrusion of liposomes. In still other embodiments, cannabidiol may be combined with a protein such as human serum albumin to act as a carrier molecule. Each of these materials added together with cannabidiol to aqueous mixtures or suspension will be referred to as co-solubilizers.

The benefit of the co-solubilization of cannabidiol is that more cannabidiol may be delivered in a substantially aqueous mixture. A further benefit is that the co-solubilized cannabidiol may gradually extract from its co-solubilizer, making the cannabidiol available in solution over an extended time. A still further benefit that the co-solubilized material acts as a reservoir to “buffer” the cannabidiol concentration in the aqueous phase to a relatively constant sustained value.

Cannabidiol suppresses interleukin (IL) 8 and 10 production and induces lymphocyte apoptosis in vitro. It is a strong inhibition of neutrophil chemotaxis and modulates tumor necrosis factor (TNF)-α, IL-1, and interferon (IFN)-γ by mononuclear cells and the suppression of chemokine production by human B cells. Cannabidiol's overall effect is generally considered anti-inflammatory, though its suppression of the anti-inflammatory IL-10 suggests more complex effects. Schmuhl et al. (in Biochemical Pharmacology, 87: 3 pp 489-501 (2014)) reported an increase of mesenchymal stem cell migration by cannabidiol via activation of p42/44 MAPK. Migration and differentiation of mesenchymal stem cells (MSCs) are known to be involved in various regenerative processes such as bone healing. Cannabidiol was reported to increase the migration of adipose-derived MSCs in a time-and concentration-dependent manner. Endocannabinoid (eCB) signaling has also been shown to regulate proliferation and differentiation of mesoderm-derived hematopoietic and mesenchymal stem cells, with a key role in determining the formation of several cell types in peripheral tissues, including blood cells, adipocytes, osteoblasts/osteoclasts and epithelial cells. Long-term stimulation with cannabidiol induced differentiation of MSCs into the osteoblastic lineage as evidenced by increased mineralization. Cannabidiol may therefore recruit MSCs to sites of calcifying tissue regeneration and subsequently support bone regeneration.

Applicant has found that cannabidiol has beneficial effects when applied in a treatment composition including either stem cells or conditioned media recovered from growing cultures of stem cells. The effects may be two-fold: first cannabidiol has direct effects on the treated tissue at various stages of the healing process. This includes reduction of inflammation, recruitment of endogenous stem cells, and support of differentiation of stem cells into end-stage cells that rebuild or remodel tissue. Second, cannabidiol may act on the stem cells of the treatment composition to alter their spectrum of cell products that improves the healing effects of these cell products. This second action of cannabidiol may occur either within the administered cells when such cells are part of a treatment composition, or it may occur within a cell culture causing the cultured cells to produce an altered spectrum of cell products that are delivered to the conditioned medium of other treatment compositions.

Cannabidiol may be admixed with culture medium to produce a modified condition medium, or it may be mixed with a treatment composition, or both.

### Stem Cells

A stem cell is an undifferentiated or relatively undifferentiated cell that is capable of giving rise to more cells of the same type, and from which certain other kinds of cell arise by differentiation. There are a variety of human stem cells that serve as reservoirs for recovery and replacement of damaged tissue. Some may be characterized by surface markers such as the above discussed Lgr6 bearing stem cells associated with interfollicular epidermis and sebaceous glands, the Lgr5 bearing follicular stem cells, and the separate Lgr6 bearing nail stem cells. Many other stem cell types, such as CD34 bearing hematopoietic stem cells, are known in the art. Some stem cells are difficult to harvest, but others are more readily available. For example, mobilized peripheral blood, umbilical cord blood, endothelium, adipose tissue, deciduous tooth mesenchyme, umbilical cord mesenchyme, and postpartum placenta may be collected without invasive procedures: all have all been reported as sources of stem cells.

The cells harvested as described below may not themselves be stem cells, but the harvest and subsequent processing conditions may induce some of the harvested cells to become stem cells.

Adipose-derived stem cells (ADSCs) are stem cells extracted from adipose tissues. Human adipose tissue is available ex vivo as a result of cosmetic procedures including liposuction. ADSCs resemble bone marrow mesenchymal stromal cells (BMSCs): ADSCs interact with endothelial cells by expressing similar integrins to BMSCs; and, like BMSCs, ADSCs lack the dominant ligand for P-selectin. However, unlike BMSCs, providers can harvest large numbers of ADSCs with low donor-site morbidity.

Harvesting from peripheral blood. Peripheral blood may be collected through conventional venipuncture. In some instances, it may be desirable to first administer colony stimulating factors such as G-CSF (filgrastim) to increase stem cell production and mobilize stem cells from the bone marrow into the peripheral circulation. The collected cells may be fractionated by centrifugation and purified by immunomagnetic separation for CD45⁺ cells as described in Balduini et al. in PLoS ONE 6(6): e21015 (2011) or for CD34⁺ as described in Spohn et al. in Cytotherapy, 2015; 17: 1465-1471 using the Prodigy and CliniMACS Plus from Miltenyi Biotec. Both publications are hereby incorporated by reference for their disclosure of fractionation and purification of stem cells.

Harvesting from liposuction aspirates. Adipose-derived stem cells (ASDCs) are stem cells extracted from adipose tissues. Adipose tissue, like other tissue types, is not a homogenous mixture of a single cell type. Instead adipose tissue includes a combination of fat cells, vasculature, connective tissue, and blood cells. Human adipose tissue is available ex vivo as a result of cosmetic procedures including liposuction.

Stem cells may be extracted from adipose tissue by any of a number of methods known in the art, including treatment with surfactants or enzymes (including proteases such as collagenase, or trypsin), maceration, separation by centrifugation, filtering, or settling, ultrasonic treatment, adherent culturing, or some combination of these methods. Immuno-magnetic separation may also be effective. Aspirated specimens from liposuction procedures (lipoaspirates) are largely composed of fat cells but also include supporting cells such as those comprising vasculature. Vascular tissue includes at least some cells identifiable as adult stem cells. Such cells bear stem cell surface markers characteristic of stem cells and detectable by flow cytometry. These cells are also capable of adherent growth in cell culture. Once extracted from adipose tissue, adipose-derived stem cells may be grown in culture by a number of methods known in the art, including growth on three-dimensional scaffolds or supports, growth in suspension culture, or growth on the surface of plastic or glass vessels.

Rodriguez et al. U.S. Pat. No. 7,531,355 B2 describes one protocol for collection and isolation of ADSCs. A hollow blunt-tipped cannula is introduced into the subcutaneous space through an about 1 cm incision. A gentle suction applied as the cannula moves through the adipose compartment mechanically disrupts and collects the fat tissue. A solution of saline and epinephrine (a vasoconstrictor) infuses into the adipose compartment to minimize blood loss and blood cell contamination of the tissue. Lipoaspirates are washed extensively with equal volumes of phosphate buffered saline (PBS) and the extracellular matrix (ECM) is digested at 37° C. for 30 minutes with 0.075% collagenase. In other embodiments, the enzymatic step may be replaced by sonication as described in Victor U.S. Pat. No. 8,440,440 B2. Both publications are hereby incorporated by reference for their disclosure of collection and isolation of ASDCs.

After treatment the dissociated adipose tissue may be centrifuged. The pellet contains the stromal vascular fraction cells (SVFCs), which include ASDCs. The SVFCs may be resuspended and further washed in PBS and plated to tissue culture bottles with Dulbecco's Modified Eagle's Medium. The plated cultures may be washed to remove non-adherent cells and incubated at 37 Celsius with 5% CO₂. Remaining growing cells include ASDCs, which may be passaged to further enrich the division competent cells.

Once harvested, stem cells can be at least partially purified by a number of methods. These methods include marker-based cell sorting (including by flow cytometric cell sorters or by solid phase affinity separation such as immunomagnetic beads), differential centrifugation, cell culturing with serial passaging, or dilution-based clonal isolation.

ADSCs display multipotentiality: they have the capability of differentiating at least along the adipocyte, chondrocyte, myogenic, neuronal, and osteoblast lineages. Rodriguez et al. (cited above) describes induction of multiple lineages of fate-directed cells from ASDCs. These include leiomyogenic phenotypes (including functional smooth muscle cells) from ASDCs by growth in a medium containing a transition material including 100 U/mL of heparin. Rodriguez et al. also describes techniques for production of myogenic phenotypes (by exposure to transition material including hydrocortisone in serum-rich media), of adipogenic phenotypes (by exposure to transition material including 10 μM insulin in combination with about 1 μM dexamethasone), of osteogenic phenotypes (by exposure to transition material including 1 μM dexamethasone and about 20 μM ascorbate-2-phosphate and between about 20 nM β-glycerophosphate), and neurogenic (or other ectodermal) phenotype (by exposure to transition material including 10 mM β-mercaptoethanol without serum).

Lis et al. in Nature Published online 17 May 2017 reported conversion of adult endothelium to immunocompetent hematopoietic stem cells in mice. The process used transient expression of the transcription-factor-encoding genes and vascular-niche-derived angiocrine factors. The transduced endothelial cells committed to a hematopoietic fate. This conversion in vitro suggests that a similar conversion may occur in vivo given the appropriate microenvironment and mix of cell products. Without intent to be bound by theory, Applicant believes that natural healing processes may include recruitment of stem cells from other cells such as endothelium cells and differentiation of stem cells into multiple lineages. The boundary between what is and what is not a multipotent stem cell may be blurred by such recruitment, conversion, and differentiation. To take maximal advantage of healing potential requires providing the appropriate microenvironment. Applicant believes that this microenvironment may be supported by stem cell products, particularly stem cell products where the stem cells are exposed to cannabidiol to appropriately modulate the production of those products. Applicant further believes that this microenvironment may be supported by the presence of cannabidiol at the site of healing, particularly when the cannabidiol is made available through co-solubilization.

Placenta-derived stem cells (PDSCs) may be derived from post-partum placentas; no invasive procedure is necessary, since the placenta is expelled after the birth of the neonate. Yen et al. described in Stem Cells, 23: p3-9 (2005) a process wherein harvested pieces of tissue were washed several times in phosphate-buffered saline (PBS) and then mechanically minced and enzymatically digested with 0.25% trypsin-EDTA. The homogenate was subsequently pelleted by centrifugation and suspended in Dulbecco's modified Eagle's supplemented by 10% fetal bovine serum (FBS). This use of FBS may be problematic as discussed below and is preferably avoided.

In embodiments, the stem cells may be autologous to the treated person. Autologous materials are derived from the treated individual. Autologous treatment means the implantation, transplantation, infusion, or transfer of human cells or their products or progeny to the individual from whom the cells or tissue were recovered.

Production of useful human cells or cell products generally requires isolation of cells from human tissue. Human cells are subject to a variety of pathogens that may be transmissible to a recipient of such cells. Human cells also carry antigens that can cause the recipient of such cells to produce an immune response to the stem cells. Alternatively, some cells may themselves produce an immune response to the recipient. For at least these reasons, autologous cells (those derived from tissues of the recipient) may be favored over cells from others. Autologous cells are not amenable to large scale manufacturing processes because each isolate is only applicable to a single individual.

As mentioned, tissue-derived products, particularly those derived from minimally processed human tissue, may contain infectious agents or antigens. The invention includes compositions containing cells derived from stem cells which may be autologous with the treated individual. Such autologous compositions are not necessarily free of infectious agents or antigens, but the treated individual has already been exposed to these autologous agents and antigens so that the risk of further infection or immune reaction should be low.

### Stem Cell Culture

Once extracted from tissue, stem cells may be grown in tissue culture by a number of methods known in the art, including growth on three-dimensional scaffolds or supports, growth in suspension culture, or growth on the surface of plastic or glass vessels.

Culture of cells for human use must take place under sterile conditions with particular attention paid to avoiding contamination by infectious organisms, including those infecting the stem cell donors. Contamination may also arise from culture materials: for example, use of serum products, such as bovine fetal calf serum (FCS), is very common in cell culture. Serum products contain an uncontrolled mixture of components that may interact with stem cells. For example, FCS may contain variable amounts of endocannabinoids such as anandamide or other ligands capable of activating the peripheral cannabinoid receptors. In embodiments, the invention provide treatment compositions derived from human cells that have not been cultured in the presence of or exposed to serum products.

Human cultured cells, including stem cells and their progeny, produce a variety of growth-promoting and healing materials such as growth factors, cytokines, stress proteins, and nutrients including TGF-B, PDGF, and GM-CSG, interleukins, and matrix proteins (collectively, cell products). While many of these have been identified, stem cells likely also secrete other substances either not yet known or with beneficial functions yet to be precisely identified. Some of these materials may be effective at low concentration.

Tissue-derived products, particularly those derived from minimally processed human tissue, may contain human infectious agents or antigens. The invention includes embodiments with compositions containing stem cells autologous with the treated individual. Such compositions are not necessarily free of infectious agents or antigens, but the treated individual has already been exposed to these autologous agents and antigens so that the risk of further infection or immune reaction should be low. Another object of this invention is to provide treatment compositions including autologous stem cells to aid healing with reduced risk of infection or immune reaction.

Human stem cells, such as ASDCs, produce a variety of growth-promoting and healing materials such as growth factors and cytokines. These mixtures of cell products may be harvested from cultured cells by collecting the culture medium to which such cells have been exposed.

Growth of such cultured stem cells includes supply of nutrients for the cells through provision of an aqueous culture medium. Cells grow in culture in contact with medium and extract nutrients from it. These cells also deliver to the medium products of their growth and metabolism. Among the products are the growth factors and cytokines discussed above as well as metabolic products. Conventional cell culture requires replacement of culture medium as cells use up nutrients and deliver products that may affect future cell growth. This replacement may be either continuous, with a portion of the medium removed as new medium is added, or intermittent with periodic replacement of some or all of the culture medium in a vessel. Culture medium removed after exposure to cells in culture is known as spent or conditioned medium. Culture medium removed after exposure to a particular cell type (such as ASDCs) will be referred to by a descriptor of the cell followed by the word medium. For example, culture medium removed after exposure to human ASDCS is human ASDC medium.

### Stem Cell Conditioned Medium

As discussed above, growth of cells such as adipose-derived stem cells includes supply of nutrients for the cells through provision of an aqueous culture medium. Cells grown in culture deliver to the medium cell products including growth factors and cytokines as well as metabolic products. Culture medium removed after exposure to cells includes stem cell products as well as residual components of the original medium, such as essential amino acids, salts, vitamins, minerals, trace metals, sugars, lipids, and nucleosides. Cell culture medium attempts to supply the components necessary to meet the nutritional needs required to grow cells in a controlled, artificial and in vitro environment. Nutrient formulations, pH, and osmolarity may vary depending on the type of cell cultured, on cell density, and on the culture system employed. The scientific literature includes description of many cell culture medium formulations; a number of such media are commercially available. Conditioned medium also contains a variety of cellular metabolites and secreted proteins, including, for example, biologically active growth factors, inflammatory mediators and other extracellular proteins. Examples of suitable culture media are Dulbecco's Modified Eagle's Medium and RPMI 1640. Such media may be supplemented by other nutrients, growth supporting materials, or antibiotics as is known in the art.

In some embodiments, the culture medium also includes cannabidiol, which may co-solubilized as described above. The presence of cannabidiol in the culture medium alters the stem cells such that their production of cytokines and other cell products is different than if the cannabidiol were not present. Applicant believes that this alteration in the “spectrum” of cell products produced by stem cells exposed to cannabidiol results in a mix of cell products in the condition medium that better promotes healing of tissue. Without intent to be bound by theory, Applicant believes that cannabidiol interacts with receptors on the stem cells and/or with enzymes within the cells so as to alter the expression of different cell products.

In some embodiments, conditioned medium may also contain added ingredients such as antimicrobials, added growth factors, peptides, or acellular extracellular matrix.

### Compositions (Prospective Examples)

In one embodiment, the invention includes a composition combining a living human stem cell with cannabidiol. The cannabidiol may be co-solubilized with a protein or a lipophilic surfactant. The lipophilic surfactant may encapsulate the cannabidiol in a liposome. In other embodiments, the cannabidiol may be co-solubilized with a protein, such as human serum albumin. In other embodiments, the cannabidiol may be dissolved in an aqueous carrier such as phosphate buffered saline.

In another embodiment, the invention includes a composition combining a conditioned medium form a culture of human stem cells. The conditioned medium is mixed with cannabidiol. The cannabidiol may be co-solubilized with a protein or a lipophilic surfactant. The lipophilic surfactant may encapsulate the cannabidiol in a liposome. In other embodiments, the cannabidiol may be co-solubilized with a protein, such as human serum albumin. In other embodiments, the cannabidiol may be dissolved in an aqueous carrier such as phosphate buffered saline. In some embodiments, the conditioned medium may be produced by a culture of human stem cells that are cultured in the presence of cannabidiol.

### Administration (Prospective Examples)

The invention includes a method of treating an affected part of the body by applying one or more compositions of the invention. The compositions may be applied to a region of the body to be treated either topically or by injection.

Direct delivery of therapeutic compositions to affected tissue may be preferred over the systemic delivery of such compositions for several reasons related to concentration of the compositions. A substantially greater concentration of such compositions can be delivered directly into affected tissue, compared with the dilute concentrations possible through systemic delivery. Systemic administration may be associated with systemic toxicity at doses required to achieve effective concentrations in the affected tissue. Further, autologous materials are necessarily available in relatively small amounts; direct delivery limits the amounts of materials needed.

Direct delivery generally means topical administration or injection into the impaired tissue. It is difficult to repair damage and address such loss of function by compositions applied topically to skin or mucosa, which is impervious to most aqueous materials. Some agents may traverse skin or mucosa to affect tissue, but such traversal is usually limited to small molecules. External topical application also does not allow exposure of deeper tissues without exposure of surface tissues. However, in some embodiments, such as treatment of superficial layers of the skin, topical administration is effective because the treatment area is directly exposed. Topical application may also be appropriate when the treatment area is exposed through a wound or other injury, or during a surgical procedure. In such embodiments, an effective amount of the composition is applied to the damaged or dysfunctional area. The compositions of the invention may be modified for topical application to keep the material in place, such as by addition of thickening agents such as methyl cellulose. In other embodiments, the composition may be applied topically to an affected area and covered with a dressing that retains the composition in place.

In some embodiments, injection of the treatment composition may be performed by loading the composition into a syringe having a hollow needle, inserting the tip of the needle into the desired treatment area, injecting a volume of the composition into the tissue, then withdrawing the needle and advancing to another treatment area. The choice of treatment areas depends on the nature and extent of the impaired tissue.

Treatment may be accompanied by a topical anesthetic such as lidocaine.

Alternatively anesthetic or analgesic function may be served or potentiated by cannabidiol if the cannabidiol is accessible at appropriate concentration to peripheral neuron receptors. CB₂ receptors have been shown to modulate acute pain (see, for example, Whiteside et al. Curr. Med. Chem 14(8) pp 917-36, 2007, incorporated by reference for its disclosure of cannabinoid receptors in relation to pain). Cannabidiol can act as either an agonist or antagonist of CB₂ receptors and so may have an effect on pain perception depending on its available concentration. Cannabidiol, as discussed above, is only marginally soluble in aqueous materials unless co-solubilized. Applicant believes that the provision of cannabidiol in co-solubilized form makes the material able to be delivered to CB₂ receptors of peripheral nerve terminals in sufficient concentration to potentiate pain relief at the site of its application.

To the extent that injury, healing, or treatment may result in pain at the site, cannabidiol admixed with stem cells or stem cell conditioned media may serve to reduce such pain because of a combination of anti-inflammatory and pain-relieving properties. In addition to its direct effect on pain perception, cannabidiol may act in concert with added stem cells to further reduce inflammation. Certain inflammatory cytokines (such as IL-1β, IL-6, and TNF-α) are involved in the process of pathological pain through nerve-injury and inflammation-induced central sensitization. As discussed above, the action of cannabidiol suppresses at least some of these cytokines, and consequently its administration may reduce such pain. Further, the action of cannabidiol on administered stem cells may alter the production of cell products from those cells so as to further reduce pain attributable to administration of the stem cells. Similarly, the action of cannabidiol on cultured stems cells may cause those cells to deliver to conditioned media a different mix of cell products than would be produced absent the presence of cannabidiol. The conditioned media so produced may further reduce the pain of injury, inflammation, and repair.

Without intent to be bound by theory, applicants believe that endogenous regenerative cells have a limited ability to restore all function to impaired tissue, particularly where the impairment is age-related. Reduced blood flow and vascular supply to the impaired region and declining hormone levels may further inhibit recuperative mechanisms. The provision of more adequate perfusion may facilitate more complete recovery by endogenous regenerative cells. Exogenous stem cells or stem cell products of the composition may participate in healing by increasing the rate of collagen secretion, vascular in-growth and fibroblast proliferation. Improved vascular ingrowth may further potentiate healing of an affected area by giving better vascular access.

Applicant has also discovered that the disclosed compositions may advantageously aid in loss of weight in obese or overweight patients, particularly when the compositions are taken systemically.

The compositions of the invention delivers it payload of tailored growth factors, which may serve to restore the activity of endogenous regenerative cells and may further serve to stimulate introduced stem cells or to recruit and convert new stem cells during treatment. These cells contribute to recovery and repair of the impaired tissues and restoration of full function.

I refer in this specification to embodiments as “one embodiment,” “an embodiment,” “another embodiment,” etc. This specification discloses various aspects of the invention with reference to particular embodiments, but it should be understood that any of the features, functions, materials, or characteristics may be combined with any other of the described features, functions, materials, or characteristics. The description of particular features, functions, materials, or characteristics in connection with a particular embodiment is exemplary only; it should be understood that it is within the knowledge of one skilled in the art to include such feature, structure, or characteristic in connection with other embodiments whether or not explicitly described. I intend the scope of the appended claims to encompass such alternative embodiments. Variations on these described embodiments will become apparent to those of ordinary skill in the art upon reading the description. The inventor expects skilled artisans to employ such variations as appropriate, and the inventors intend for the invention to be practiced otherwise than specifically described herein. Accordingly, this specification and claims include all modifications and equivalents of the subject matter recited in the claims appended hereto as permitted by applicable law.

Unless otherwise indicated, all numbers used in the specification and claims are to be understood as being modified in all instances by the term “about.” Unless indicated to the contrary, the numerical values in the specification and attached claims are approximations that may vary depending upon the desired properties sought to be obtained.

I claim:

1. A treatment composition comprising:
a human stem cell;
a first amount of cannabidiol; and
an aqueous carrier.

2. The composition of claim 1, wherein the human stem cell includes one or more of a hematopoietic stem cell, an endothelium-derived stem cell, adipose-derived stem cell, a tooth mesenchyme-derived stem cell, or a placenta-derived stem cell.

3. The composition of claim 1, wherein the cannabidiol is associated with a co-solubilizing agent.

4. The composition of claim 3, wherein the co-solubilizing agent includes one or more of a lipophilic surfactant or a protein.

5. The composition of claim 3, wherein the co-solubilizing agent includes one or more of dipalmitoylphosphatidylcholine or phosphatidylinositol.

6. The composition of claim 5, wherein the cannabidiol is packaged in a liposome.

7. A treatment composition comprising:
a conditioned medium from a human stem cell culture; and
a first amount of cannabidiol.

8. The composition of claim 7, wherein the conditioned medium is derived from a culture admixed with a second amount of cannabidiol.

9. The composition of claim 8, wherein the second amount of cannabidiol is associated with a co-solubilizing agent.

10. The composition of claim 7, wherein the human stem cell culture includes one or more of a hematopoietic stem cell, an endothelium-derived stem cell, adipose-derived stem cell, a tooth mesenchyme-derived stem cell, or a placenta-derived stem cell.

11. The composition of claim 7, wherein the first amount of cannabidiol is associated with a co-solubilizing agent.

12. The composition of claim 11, wherein the co-solubilizing agent includes one or more of a lipophilic surfactant or a protein.

13. The composition of claim 12, wherein the co-solubilizing agent includes one or more of dipalmitoylphosphatidylcholine or phosphatidylinositol.

14. The composition of claim 13, wherein the cannabidiol is packaged in a liposome.

15. A method of treating a body dysfunction comprising administering the composition of claim 1 or 9 to an affected area of a human.

16. The method of claim 15, wherein the step of administering includes a topical application of the composition to the affected area.

17. The method of claim 16, wherein the composition further includes a thickening agent.

18. The method of claim 15, wherein the step of administering includes an injection of the composition to the affected area.
